(19) United States
(12) Patent Application Publication
(10) Pub. No.: US 2023/0268045 A1
(43) Pub. Date: Aug. 24, 2023

(54) GENERATION OF ANALYTICS

(71) Applicant: Insight RX, Inc., San Francisco, CA (US)

(72) Inventors: Srijib Goswami, Walnut Creek, CA (US); Ron Keizer, Lafayette, CA (US)

(21) Appl. No.: 17/680,265

(22) Filed: Feb. 24, 2022

(51) Int. Cl. G16H 20/10; G16H 10/60; G16B 5/00; G16B 5/30

(52) U.S. Cl. CPC G16H 20/10; G16H 10/60; G16B 5/00; G16B 5/30

(57) ABSTRACT

Various methods, systems and computer program products of the Parameter Identification Engine receives a first batch of patient data. The Parameter Identification Engine identifies a plurality of sets of estimated input parameters for a pharmacology model(s) based on an estimated or assumed uncertainty distribution for the input parameters. The Parameter Identification Engine generates one or more patient objective function values, whereby each respective patient objective function value represents a confidence measure that a corresponding set of estimated input parameters fed into the pharmacology model returns model output comprising one or more values in the received patient data. In some embodiments, the values in the received patient data may be based on measured and observed actual data from patients.

### BACKGROUND

Various characteristics of a patient may influence the extent of the patient's response to a medication. Current conventional systems rely on simplified dosing guidelines, and in certain cases, a static pharmacology model. Individualized patient dosing of a medication is essential for a desired therapeutic effect. Patients receiving complex medications benefit from optimal dosing and conventional technology that assists doctors and clinicians in determining the proper dosing for each patient is still limited in effectiveness.

### SUMMARY

Conventional systems are deficient with respect to collecting and aggregating vast amounts of patient data from multiple remote sources and continually retraining and optimizing multiple varying types of statistical customized drug dosing models without the need to continually access, format and reprocess portions of patient data that were already previously used in prior training and optimization phases. Such continual access and reprocessing of previously used patient data creates a significant storage and computational burden.

According to various embodiments, a remote dosing system may execute a prediction model(s) for determining the behavior, results and effects of various medication dosages on one or more patients. According to an embodiment, a prediction model executed by the remote dosing system may rely on a set of estimate patient input parameters in order to generate predicted dosage output. Various embodiments of a Parameter Identification Engine as described herein are implemented to identify an accurate set of estimated input parameters that can be pushed out (i.e. transmitted) to the remote dosing system. As such, the Parameter Identification Engine continually updates the remote dosing system's set of estimate patient input parameters used by its prediction model(s).

Various embodiments of the remote dosing system include an analytics dashboard for the filtering, sorting and visualization of various types and subsets patient data. In addition, the analytics dashboard includes visualizations of output and predictions of one or more drug dosing models. The analytics dashboard of the remote dosing system generates and displays a user interface to an end-user at the remote source.

Various methods, systems and computer program products of the Parameter Identification Engine receives a first batch of patient data. The Parameter Identification Engine identifies a plurality of sets of estimated input parameters for a pharmacology model(s) based on an estimated or assumed uncertainty distribution for the input parameters. The Parameter Identification Engine generates one or more patient objective function values, whereby each respective patient objective function value represents a confidence measure that a corresponding set of estimated input parameters fed into the pharmacology model returns model output comprising one or more values in the received patient data. In some embodiments, the values in the received patient data may be based on measured and observed actual data from patients.

The Parameter Identification Engine generates an importance ratio weight (or importance ratio) for each set of estimated input parameters, each respective importance ratio weight represents a corresponding set's performance and accuracy with respect to the first batch of data. The Parameter Identification Engine updates the posterior distribution for the input parameters and identifies a most accurate set of estimated input parameters based on the importance ratio weights and sends the identified set of estimated input parameters to a remote dosing model system. By sending the identified set of estimated input parameters, the Parameter Identification Engine provides an input parameter update to the one or more pharmacology model implemented by the remote dosing model system.

According to various embodiments, the Parameter Identification Engine continually receives batches of patient data. For example, patient data may include observed patient data representing medication administration time, dosing information, patient demographics, clinical labs etc.). Upon receiving a subsequent batch of patient data, the Parameter Identification Engine updates the one or more patient objective function values, whereby each respective updated patient objective function value represents a confidence measure that a corresponding set of estimated input parameters fed into the pharmacology model returns model output comprising one or more values present in the newly received patient data. The Parameter Identification Engine aggregates all the patient objective function values for each patient—across all batches of patient data—to calculate a cumulative objective function value for each set of estimated input parameters.

The Parameter Identification Engine updates each set's cumulative importance ratio weight based on that set's newly generated cumulative objective function value. A set's importance ratio weight thereby acts as a store of that set's precision and accuracy across all patient's in each batch of patient data. By generating and updating the cumulative importance ratio weight, the Parameter Identification Engine provides the advantage of providing functionality for continually evaluating the posterior distribution of the input parameters, and identifying an updated set of estimated input parameters as each batch of patient data is received—without having to access and re-process previously received batches of patient data.

According to some embodiments, the Parameter Identification Engine may continually receive various subsets of new and/or updated patient data from one or more remote sources.

In some embodiments, one or more software modules of the remote dosing system may be implemented and executing at a remote source, such as a hospital institution.

One or more software modules of the Parameter Identification Engine may be implemented and executing on a cloud computing platform for continuous learning operations and calculations as well as continuous retraining and optimization of multiple differing types of drug dosing models (“models”). For example, the various types of models may be one or more types of population pharmacokinetic models represented according to one or more machine learning techniques.

Various embodiments include a module(s) and/or one or more functionalities to redact privacy information/data, to encrypt information/data and to anonymize data to ensure the confidentiality and security of user and platform information/data as well as compliance with data privacy law(s) and regulations in the United States and/or international jurisdictions.

Further areas of applicability of the present disclosure will become apparent from the detailed description, the claims and the drawings. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1A is a diagram illustrating an exemplary environment in which some embodiments may operate;

FIG. 1B is a diagram illustrating an exemplary environment in which some embodiments may operate;

FIG. 2A is a diagram illustrating an exemplary environment in which some embodiments may operate;

FIG. 2B is a diagram illustrating an exemplary environment in which some embodiments may operate;

FIG. 3 is a diagram illustrating an exemplary method that may be performed in some embodiments.

FIG. 4 are each a diagram illustrating data that corresponds with an exemplary environment in which some embodiments may operate;

FIG. 5 is a diagram illustrating an exemplary environment in which some embodiments may operate.

FIG. 6 is a diagram illustrating an exemplary environment in which some embodiments may operate.

FIG. 7 is a diagram illustrating an exemplary environment in which some embodiments may operate.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

A diagram of exemplary network environment in which embodiments may operate is shown in FIG. IA. In the exemplary environment 140, two clients 141, 142 are connected over a network 145 to a server 150 having local storage 151. Clients and servers in this environment may be computers. Server 150 may be configured to handle requests from clients.

The exemplary environment 140 is illustrated with only two clients and one server for simplicity, though in practice there may be more or fewer clients and servers. The computers have been termed clients and servers, though clients can also play the role of servers and servers can also play the role of clients. In some embodiments, the clients 141, 142 may communicate with each other as well as the servers. Also, the server 150 may communicate with other servers.

The network 145 may be, for example, local area network (LAN), wide area network (WAN), telephone networks, wireless networks, intranets, the Internet, or combinations of networks. The server 150 may be connected to storage 152 over a connection medium 160, which may be a bus, crossbar, network, or other interconnect. Storage 152 may be implemented as a network of multiple storage devices, though it is illustrated as a single entity. Storage 152 may be a file system, disk, database, or other storage.

In an embodiment, the client 141 may perform the method 200 or other method herein and, as a result, store a file in the storage 152. This may be accomplished via communication over the network 145 between the client 141 and server 150. For example, the client may communicate a request to the server 150 to store a file with a specified name in the storage 152. The server 150 may respond to the request and store the file with the specified name in the storage 152. The file to be saved may exist on the client 141 or may already exist in the server's local storage 151. In another embodiment, the server 150 may respond to requests and store the file with a specified name in the storage 151. The file to be saved may exist on the client 141 or may exist in other storage accessible via the network such as storage 152, or even in storage on the client 142 (e.g., in a peer-to-peer system).

In accordance with the above discussion, embodiments can be used to store a file on local storage such as a disk or on a removable medium like a flash drive, CD-R, or DVD-R. Furthermore, embodiments may be used to store a file on an external storage device connected to a computer over a connection medium such as a bus, crossbar, network, or other interconnect. In addition, embodiments can be used to store a file on a remote server or on a storage device accessible to the remote server.

Furthermore, cloud computing is another example where files are often stored on remote servers or remote storage systems. Cloud computing refers to pooled network resources that can be quickly provisioned so as to allow for easy scalability. Cloud computing can be used to provide software-as-a-service, platform-as-a-service, infrastructure-as-a-service, and similar features. In a cloud computing environment, a user may store a file in the “cloud,” which means that the file is stored on a remote network resource though the actual hardware storing the file may be opaque to the user.

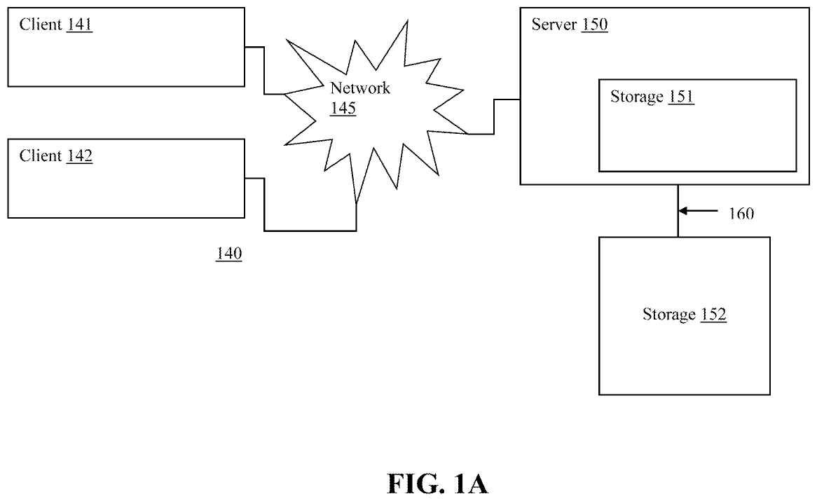

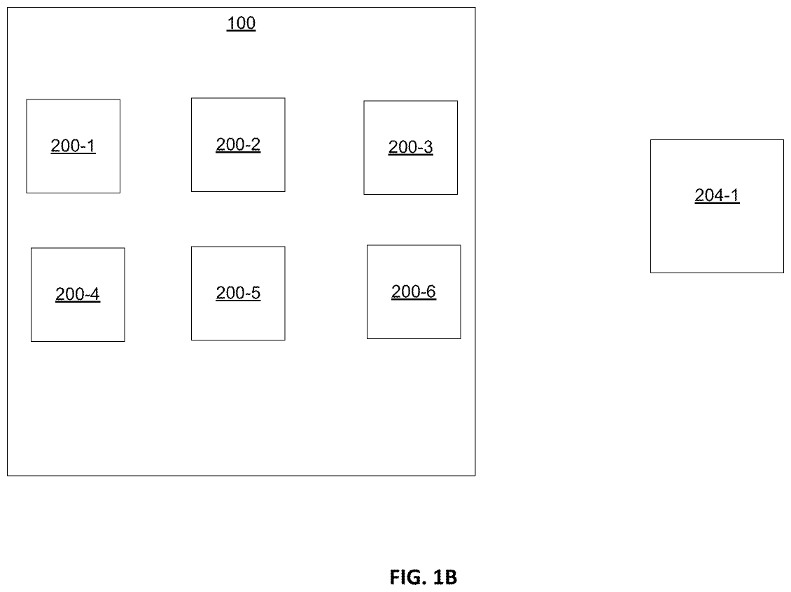

FIG. 1B illustrates a block diagram of an example system 100 for a Parameter Identification Engine that includes a batch receipt module 200-1, a set identification module 200-2, an objective function value generation module 200-3, an importance ratio weight generation module 200-4, a parameter update identification module 200-5, an update transmission module 200-6 and a user interface (U.I.) module 204-1. The system 100 may communicate with a user device 140 to display output, via a user interface 144 generated by an application Parameter Identification Engine 142.

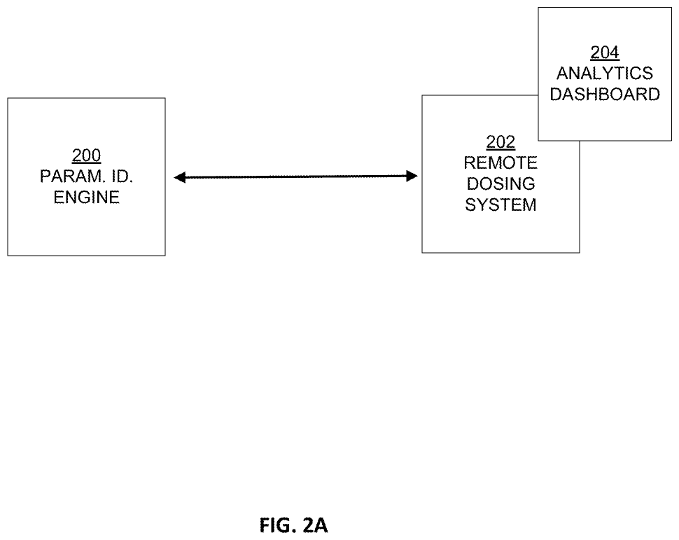

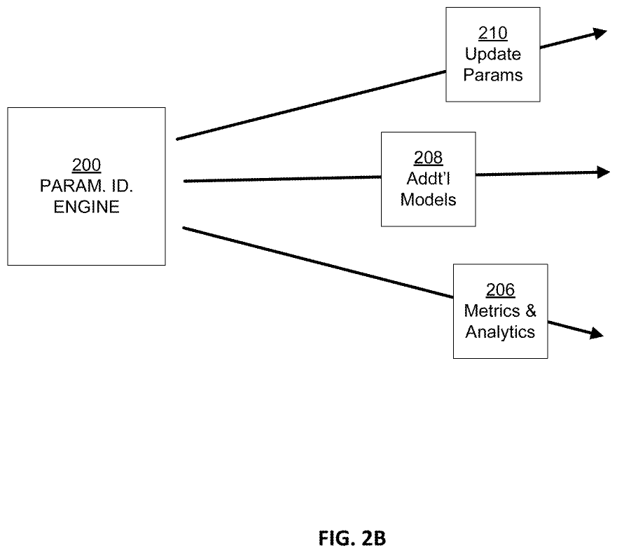

The batch receipt module 200-1 of the system 100 may perform functionality as illustrated in FIGS. 2, 3, 4, 5 and/or 6 (“FIGS. 2-6”). In some embodiments, the module 200-1 may perform associated with functionality for receiving one or more batches of patient data.

The set identification module 200-2 of the system 100 may perform functionality illustrated in FIGS. 2-6. In some embodiments, the module 200-2 may perform associated with functionality for identifying a plurality of sets of estimated input parameters.

The objective function value generation module 200-3 of the system 100 may perform functionality illustrated in FIGS. 2-6. In some embodiments, the module 200-3 may perform associated with functionality for generating one or more patient objective function values (i.e. individual objective function values).

The importance ratio weight generation module 200-4 of the system 100 may perform functionality as illustrated in FIGS. 2-6. In some embodiments, the module 200-4 may perform associated with functionality for generating one or more importance ratio weights.

The parameter update identification module 200-5 of the system 100 may perform functionality as illustrated in FIGS. 2-6. In some embodiments, the module 200-5 may perform associated with functionality for identifying a most accurate set of estimated input parameters based on the generated pharmacology model output.

The update transmission module 200-6 of the system 100 may perform functionality as illustrated in FIGS. 2-6. In some embodiments, the module 200-6 may perform associated with functionality for transmitting updated input parameters to one or more remote dosing model systems.

The user interface module 204-1 may be implemented on a remote dosing system for generating an analytics dashboard as discussed herein with respect to in FIGS. 2-6.

As shown in FIG. 2A, the Parameter Identification Engine 200 is connected to, and communicates with, a remote dosing system 202. The Parameter Identification Engine 200 includes various modules 200-1, 200-2, 200-3, 200-4, 200-5, 200-6. The remote dosing system 202 includes an analytics dashboard 204 and a corresponding module 204-1. According to various embodiments, the analytics dashboard 204 receives a selection(s) from an end-user indicating one or more requests to sort and filter patient data. For example, the end-user may access the analytics dashboard 204 to assess the accuracy of a default dosing model, implemented by the remote dosing system 202, with regard to data of a selected subset of a patient population. A default model may be, for example, a particular type of drug dosing model selected by a respective end-user. The selected subset of the patient population may be, for example, elderly patients within an age range that have a diagnosis that indicates a certain degree of organ dysfunction.

The Parameter Identification Engine 200 receives dashboard requests related to the end-user's activity and accesses precalculated and continually updated analytics and metrics that correspond to the selected patient population subset. As shown in FIG. 2B, the Parameter Identification Engine 200 transmits the precalculated analytics and metrics 206 back to the remote dosing system 202 for rendering and display at the analytics dashboard 204. Based on the received analytics and metrics, the remote dosing system 202 provides a visualization—via the analytics dashboard 204—of a default model's precision and accuracy with respect to predicting appropriate drug dosing (and its effects) for respective patients in the selected patient population subset.

In one or more embodiments, the Parameter Identification Engine 200 further transmits one or more additional types of dosing models 208 for implementation at the remote dosing system 202. Predictive output from the additional models may further be visualized within the analytics dashboard 204. The one or more additional models may be selected by the Parameter Identification Engine 202 as respective retrained and/or optimized models that outperform the default model currently implemented at the remote dosing system 202 with respect to generating accurate and precise predictions for the selected patient population subset—or an entire patient population associated with a particular remote source.

According to various embodiments, as the Parameter Identification Engine 200 receives various batches of patient data, it may continually calculate and process output from multiple different types of dosing models. The Parameter Identification Engine 200 may further optimize and retrain those different types of dosing models and continually compare the output and accuracy the current optimized versions of the dosing models generated at the Parameter Identification Engine 200 against the real-time output of the default model (and other dosing models) implemented at the remote dosing system 202.

The Parameter Identification Engine 200 may further apply one or more accuracy thresholds to the comparisons. Upon determining, by the Parameter Identification Engine 200, that a particular optimized version of the dosing model(s) meets or exceeds and accuracy threshold, the Parameter Identification Engine 200 may send a notification to the remote dosing system 202 recommending implementation of the particular optimized version of the dosing model by the remote dosing system 202. Upon receipt of an acceptance of the recommendation notification sent from the remote dosing system 202, the Parameter Identification Engine 200 sends the particular optimized version of the dosing model to the remote dosing system 202.

The Parameter Identification Engine 200 further identifies and transmits estimated input parameters 210 that are to be utilized by one or more predictive dosing models implemented at the remote dosing system 202. According to various embodiments, a predictive dosing model may have one or more types of parameters that corresponds with input values from received patient data that are implemented to determine predicted dosages. The Parameter Identification Engine 200 sends updates to the remote dosing system 2002. The updates include updated parameters 210 for the one or more predictive dosing models implemented at the remote dosing system 202.

The Parameter Identification Engine 200 continually receives batches of patient data from various sources and processes each batch of data—on a per-batch basis—against multiple, different sets of estimated input parameters. After each batch is received, the Parameter Identification Engine 200 identifies a set of estimated input parameters as the most accurate (i.e. as the most optimal set of estimated input parameters) across all previously received batches of data without having to access and reprocess the previously received and previously processed batches of patient data. The most accurate (i.e. optimal) set of estimated input parameters is sent back to the remote dosing system as parameter updates 210 for the parameters used by the predictive dosing model(s) at the remote dosing system 202.

In various embodiments, a model at the Parameter Identification Engine 200 further includes a distribution of uncertainty weights for each set of input parameters. That is, if a model has, for example, 10 different parameters, then the model includes 10 different uncertainty weight distributions, whereby each parameter has its own corresponding distribution of uncertainty weight values. In various embodiments, an uncertainty weight in a parameter's distribution of uncertainty comprises a weight value representing a likelihood that the corresponding parameter's current value (as provided from input patient data) will result in the model generating an accurate prediction of an appropriate customized dosage for a patient.

As the Parameter Identification Engine 200 receives a new (or updated) subset(s) of patient data, the Parameter Identification Engine 200 retrains one or more models over the new subset of patient data without reprocessing any of the patient data previously used in prior training phases. By eliminating the need to access and reprocess previously used patient data, the Parameter Identification Engine 200 optimizes the efficiency and processing burden of retraining multiple models that have already been trained on a vast amount of previous input patient data.

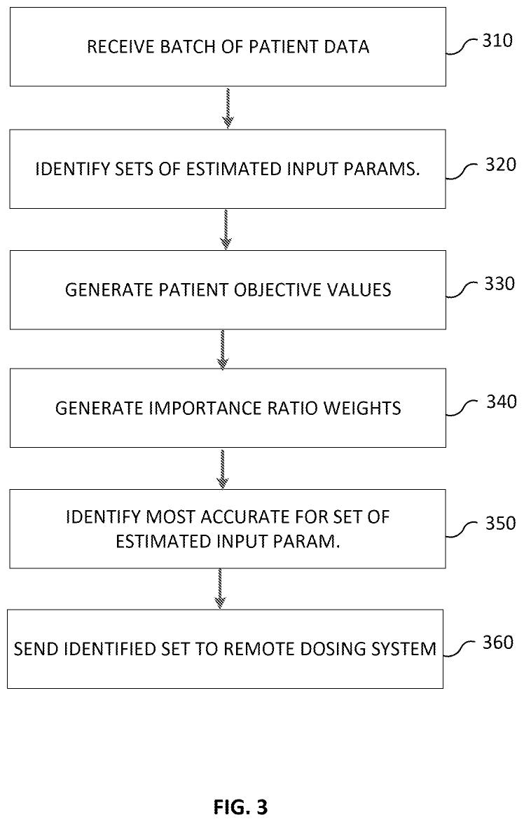

As shown in FIG. 3, the Parameter Identification Engine 200 receives a batch of patient data. (Act 310) Each batch of patient data includes known values for variables that have been actually measured or observed in one or more patients. Input variables may be patient-specific data, such as age, weight, height, dosing regimens, etc. The patient input variables further include known measured concentrations of a medication that correspond with an administered particular medication dosage amount given a particular patient's other input parameters. In other embodiments, the patient input variables may further include biomarkers such as, for example, white blood cell counts, measures of blood clotting, tumor size, growth rate of tumors, etc.

The Parameter Identification Engine 200 identifies a plurality of sets of estimated input parameters. (Act 320) According to various embodiments, such input parameters may be pre-defined model parameters that describe pharmacokinetic and/or pharmacodynamic characteristics. A set of estimated input parameters is a pre-defined set of sampled distribution values, whereby various distribution values are sourced from one or more uncertainty distributions. Therefore, a set of estimated input parameters for a particular pharmacology model is comprised of maximum likelihood estimates based on the patient input data (i.e. weight, height, previously measured output concentration data) and prior patient input parameters.

To identify a set of estimated input parameters, the Parameter Identification Engine 200 samples prior patient input parameters from the pre-defined parameter uncertainty distributions. That is, a first estimated input parameter is a maximum likelihood estimate value sampled from a first uncertainty distribution and a second estimated input parameter is thereby a parameter value sampled from a second uncertainty distribution.

The Parameter Identification Engine 200 generates an individual (or patient) objective function value for each set of estimated input parameters on a per-patient basis (Act 330). In one or more embodiments, an individual (or patient) objective function value represents the likelihood that its associated set of estimated input parameters will result in the known measured output concentration values received in a particular patient's data, given the respective set of population parameters sampled from the uncertainty distribution.

In an exemplary embodiment, for a first pharmacology model, implemented by the Parameter Identification Engine 200 with a first set of estimated input parameters, may receive a first batch of patient data that includes first patient input data and second patient input data. The first pharmacology model further includes 500 different sets of estimated input parameters. The Parameter Identification Engine 200 thereby generates a respective individual objective function value for each set of estimated input parameters per patient for that first batch of patient data. That is, the Parameter Identification Engine 200 generates 500 individual objective function values for each patient represented in the first batch due to the presence of 500 different sets of estimated input parameters.

A second batch of patient data may subsequently be received by the Parameter Identification Engine 200. Upon receipt of the second batch of patient data, the Parameter Identification Engine 200 thereby generates 500 updated individual objective function values for each patient represented in the subsequently received second batch. For example, the Parameter Identification Engine generates 500 updated individual objective function values for the first patient in response to receipt of the second batch—without accessing and reprocessing that first patient's input data from the previous first batch.

The Parameter Identification Engine 200 generates a cumulative objective function value for each set of estimated input parameters. For example, with regard to a particular set of estimated input parameters and a first pharmacology model, the Parameter Identification Engine 200 aggregates the first pharmacology model's output for each patient in each received batch of patient data. For example, if the first pharmacology model generated output for a first patient in response to a first batch of data and a second batch of data, the Parameter Identification Engine 200 aggregates that first patient's model output from both the first and the second batches of data in order to generate the cumulative objective function value for that particular set of estimated input parameters.

The Parameter Identification Engine 200 generates one or more importance ratio weights. (Act 340) An importance ratio for a given set of input parameters is computed statistically as the likelihood of the input patient data in the batch, given the set of input parameters and the pharmacological model, weighted by the likelihood of the prior set of input parameters in the distribution of the parameter sets.

All individual objective function values from an iteration are collected to calculate a weight for each set of parameters. The weight is calculated by comparing, for the respective parameter sets, the individual objective function values to the individual objective function value at the maximum likelihood across all patients.

The Parameter Identification Engine 200 identifies a most accurate set of estimated input parameters based on the generated pharmacology model output. (Act 350)

The Parameter Identification Engine 200 sends the identified set of estimated input parameters to a remote dosing model system. (Act 360) It is understood that, in various embodiments, the set of estimated input parameters is initially sampled and are then maintained for the duration of the processing described with respect to Acts 330-360.

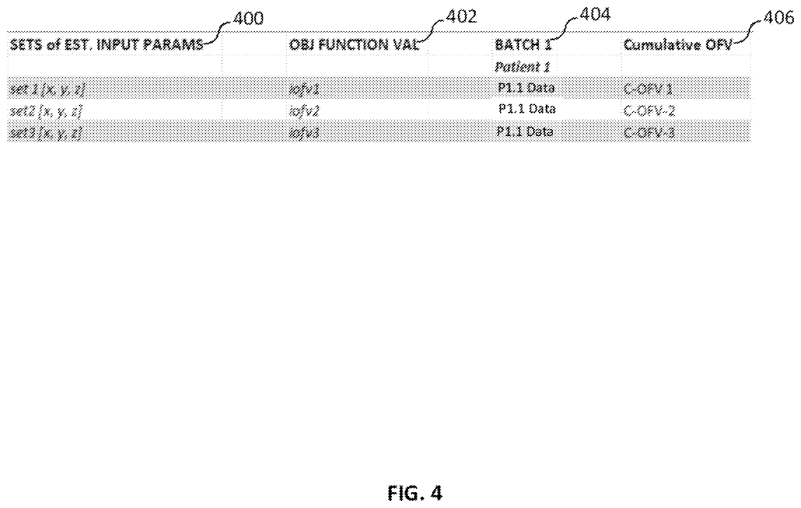

As shown in FIG. 4, a pharmacology model implemented by the Parameter Identification Engine 200 may include a plurality of sets of estimated input parameters 400. It is understood that the plurality of sets of estimated input parameter may include any number of different sets. In one embodiment, the Parameter Identification Engine 200 generates a corresponding individual objective function value 402 (iofv1, iofv2, iofv3) for each set of estimated input parameters 400 (Set1, Set2, Set3) on a per patient basis. For example, if a first batch 404 of patient data includes only a single patient, then that patient will have three different individual objective function values 402—one for each set 400. The patient data (P1.1 data) in the first batch 404 includes known concentration measurement values (concentrations 1 . . . n) and the Parameter Identification Engine 200 generates each corresponding individual objective function value 402 (iofv1, iofv2, iofv3) based on the corresponding set of estimated input parameters 400 and patient data. Each generated individual objective function value 402 (iofv1, iofv2, iofv3) is a confidence measure of how likely the model will output the known concentration measurement values by using the corresponding set of estimated input parameters 400 (Set1, Set2, Set3). The cumulative objective function value 406 will be based on the first patient's objective function values.

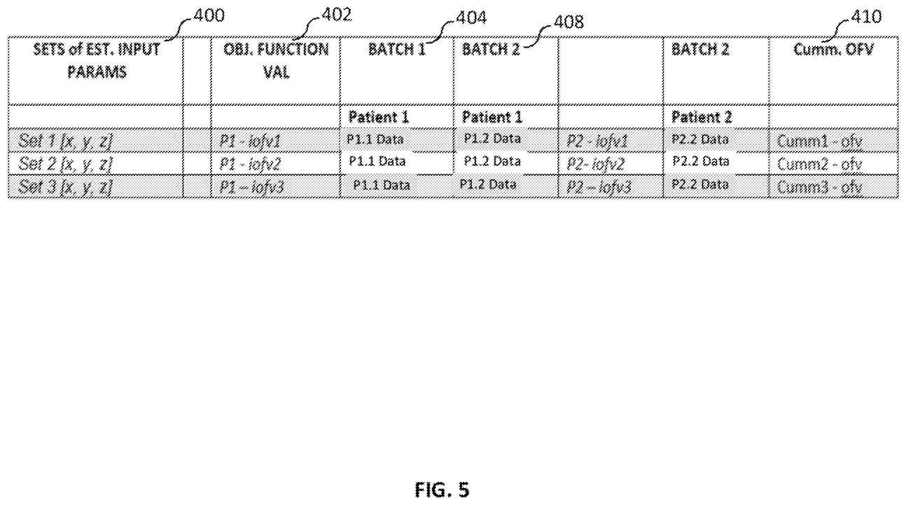

As shown in FIG. 5, the Parameter Identification Engine 200 receives a second batch of patient data 408. The second batch of patient data 408 includes additional known measured concentrations for two different patients. The Parameter Identification Engine 200 re-generates a corresponding individual objective function value 402 (P1-iofv1, P1-iofv2, P1-iofv3) for each set of estimated input parameters 400 (Set1, Set2, Set3) for the first patient. Each newly generated individual objective function value 402 (P1-iofv1, P1-iofv2, P1-iofv3) is an updated confidence measure of how likely the model will output the known concentration measurement values (concentrations x . . . y) for the first patient in the second batch of patient data (P1.2 data) by using the corresponding set of estimated input parameters 400 (Set1, Set2, Set3). It is understood that the known concentration measurement values for the first patient in the second batch (concentrations x . . . y) further include the known concentration measurement values for the first patient in the previous first batch (concentrations 1 . . . n).

The Parameter Identification Engine 200 further generates a corresponding individual objective function value 402 (P2-iofv1, P2-iofv2, P2-iofv3) for each set of estimated input parameters 400 (Set1, Set2, Set3) for the second patient. Each individual objective function value 402 (P2-iofv1, P2-iofv2, P2-iofv3) is thereby a confidence measure of how likely the model will output the known concentration measurement values (concentrations a . . . z) for the second patient in the second batch of patient data (P2.2 data) by using the corresponding set of estimated input parameters 400 (Set1, Set2, Set3).

The Parameter Identification Engine 200 recalculates a cumulative objective function value 410 for each set 400. The Parameter Identification Engine 200 aggregates all objective function values for all patients in each batch of patient data for the cumulative objective function value 410. For example, to calculate the cumulative objective function value 410 of Set 1, Parameter Identification Engine 200 aggregates the first patient's objective function values from the first and second batches 404, 408 (i.e. iofv1, P1-iofv1) along with the second patient's objective function value for the second batch 408 (i.e. P2-iofv1). It follows, then, to calculate the cumulative objective function value 410 of Set 2, Parameter Identification Engine 200 aggregates the first patient's objective function values from the first and second batches 404, 408 (i.e. iofv2, P1-iofv2) along with the second patient's objective function value for the second batch 408 (i.e. P2-iofv2).

The Parameter Identification Engine 200 utilizes the cumulative objective function values 410 to generate an importance ratio weight for each set of estimated input parameters 400 (Set1, Set2, Set3). Similar to description for a single batch of patient data, for multiple batches of data, an importance ratio for a given set of input parameters is computed statistically as the likelihood of the input patient data cumulatively over all batches, given the set of input parameters and the pharmacological model, weighted by the likelihood of the prior set of input parameters in the distribution of the parameter sets.

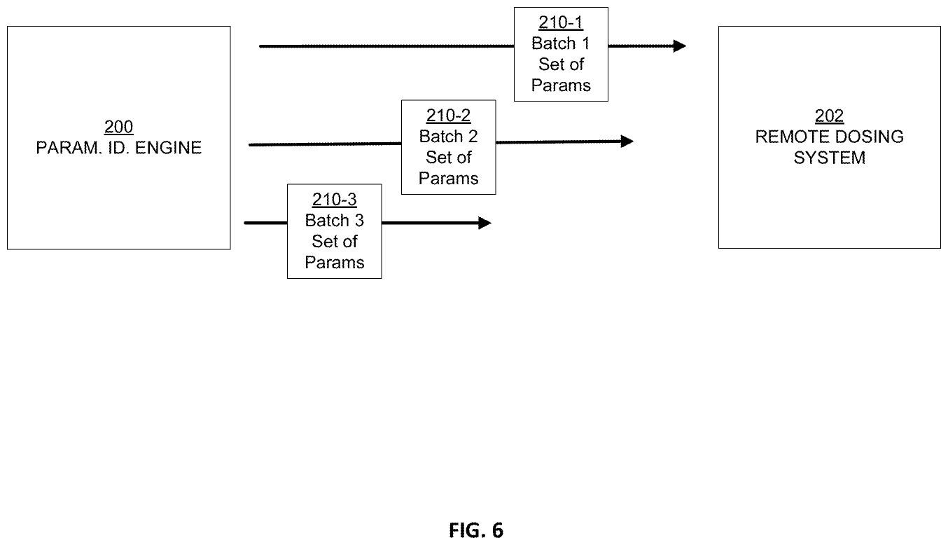

As shown in FIG. 6, as the Parameter Identification Engine 200 receives batches of patient data, it identifies a most accurate set of estimated input parameters to be pushed out to the remote dosing system 202 as parameter updates. After receiving a first batch of patient data, Parameter Identification Engine 200 calculates patient objective function values for each patient for each set, calculates the cumulative objective function values for each set, generates importance weight ratios for each set and identifies a most accurate (i.e. most optimal) set of estimated input parameters. The identified set of estimated input parameters 210-1 are sent to the remote dosing system 202 as parameter updates.

After receiving a second batch of patient data, Parameter Identification Engine 200 re-calculates the patient objective function values for each patient (identified in the second batch of patient data) for each set, re-calculates the cumulative objective function values for each set, updates the importance weight ratios for each set and identifies a most accurate set of estimated input parameters. The newly identified set of estimated input parameters 210-2 are sent to the remote dosing system 202 as parameter updates.

After receiving a third batch of patient data, Parameter Identification Engine 200 again re-calculates the patient objective function values for each patient (identified in the third batch of patient data) for each set, re-calculates the cumulative objective function values for each set, updates the importance weight ratios for each set and identifies a most accurate set of estimated input parameters. The newly identified set of estimated input parameters 210-3 are again sent to the remote dosing system 202 as parameter updates.

Various embodiments of the Parameter Identification Engine 200 and/or remote dosing system 202 may implement any suitable statistical or machine learning training techniques to train the dosing models. For example, the implemented statistical or machine learning training techniques may include, but not limited to, non-linear mixed-effects model (NLME) estimation methods based on gradient descent such as first-order conditional estimation, or sampling-based methods such as stochastic approximation expectation maximization (SAEM), a neural net based algorithm, such as Artificial Neural Network, Deep Learning; a robust linear regression algorithm, such as Random Sample Consensus, Huber Regression, or Theil-Sen Estimator; a kernel based approach like a Support Vector Machine and Kernel Ridge Regression; a tree-based algorithm, such as Classification and Regression Tree, Random Forest, Extra Tree, Gradient Boost Machine, or Alternating Model Tree; Naive Bayes Classifier; and other suitable machine learning algorithms.

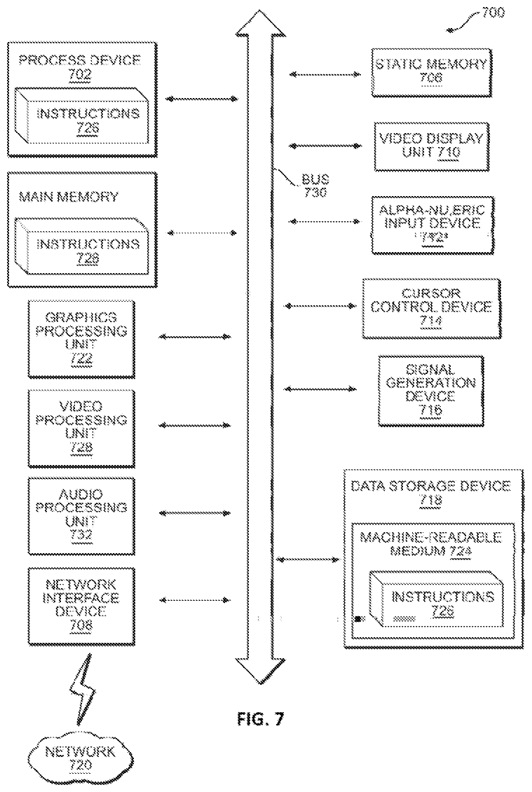

FIG. 7 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 700 includes a processing device 702, a main memory 704 (e.g., read-only memory (ROM), flash memory, dynamic random access memory (DRAM) such as synchronous DRAM (SDRAM) or Rambus DRAM (RDRAM), etc.), a static memory 706 (e.g., flash memory, static random access memory (SRAM), etc.), and a data storage device 718, which communicate with each other via a bus 730.

Processing device 702 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. The processing device 702 is configured to execute instructions 726 for performing the operations and steps discussed herein.

The computer system 700 may further include a network interface device 708 to communicate over the network 720. The computer system 700 also may include a video display unit 710 (e.g., a liquid crystal display (LCD) or a cathode ray tube (CRT)), an alphanumeric input device 712 (e.g., a keyboard), a cursor control device 714 (e.g., a mouse), a graphics processing unit 722, a signal generation device 716 (e.g., a speaker), graphics processing unit 722, video processing unit 728, and audio processing unit 732.

The data storage device 718 may include a machine-readable storage medium 724 (also known as a computer-readable medium) on which is stored one or more sets of instructions or software 726 embodying any one or more of the methodologies or functions described herein. The instructions 726 may also reside, completely or at least partially, within the main memory 704 and/or within the processing device 702 during execution thereof by the computer system 700, the main memory 704 and the processing device 702 also constituting machine-readable storage media.

In one implementation, the instructions 726 include instructions to implement functionality corresponding to the components of a device to perform the disclosure herein. While the machine-readable storage medium 724 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A computer implemented method, comprising:
identifying a plurality of sets of estimated input parameters for a pharmacology model:
receiving a first batch of patient data;
generating one or more patient objective function values, each respective patient objective function value representing a confidence measure that a corresponding set of estimated input parameters fed into the pharmacology model returns model output comprising one or more values in the received patient data;
generating an importance ratio for each set of estimated input parameters, each respective importance ratio representing a corresponding set's performance and accuracy with respect to the first batch of data;
identifying a most accurate set of estimated input parameters based on the generated pharmacology model output
sending an update set of estimated input parameters to a remote dosing model system, the update set of estimated input parameters comprising the identified set of estimated input parameters
receiving, at the remote dosing system, a selection of a type of patient population data and an identification of a particular medication;
executing the one or more prediction dosing models according to the update set of estimated input parameters, the selected type of patient population data and the identified particular medication;
generating, at the remote dosing system, a prediction of at least one behavior of the particular medication at an amount of medication dosage in the selected type of patient population; and
rendering, via a visualization dashboard of the remote dosing system, a graphical visualization of accuracy of the one or more executed prediction dosing models;
wherein generating one or more patient object function values comprises:
calculating a per-patient objective function for each patient identified in a received batch of patient data, the received batch of patient includes observed data of at least a first patient and a second patient, wherein calculating the per-patient objective function comprises:
(i) calculating a first patient objective function value for the first patient representing a first confidence measure of whether a set of estimated input parameters (“Input Set”) fed into a pharmacology model results in returned first model output comprising one or more values of the observed data of the first patient; and
(ii) calculating a second patient objective function value for the second patient representing a second confidence measure of whether the same Input Set fed into the pharmacology model results in returned second model output comprising one or more values of the observed data of the second patient;
wherein generating the importance ratio comprises:
responsive to receipt of the batch of patient data:
(a) calculating a cumulative objective function value for the Input Set based on aggregating the first and the second patient objective functions and any one or more per-patient objective functions for first and the second patient previously calculated due to receipt of one or more preceding batches of data that included earlier observed data of the first and the second patient; and
(b) generating an importance ratio weight based on the cumulative objective function value, the importance ratio weight representing the Input Set's accuracy with respect to the received batch of patient data and the preceding batches of data;
receiving a subsequent batch of patient data that includes subsequent observed data of the first patient and the second patient;
wherein generating one or more patient object function values further comprises:
calculating an update first patient objective function value for the first patient representing an update first confidence measure of whether the Input Set fed into the pharmacology model results in returned first model output comprising one or more values of the subsequent observed data of the first patient;
calculating an update second patient objective function value for the second patient representing an update second confidence measure of whether the same Input Set fed into the pharmacology model results in returned second model output comprising one or more values of the subsequent observed data of the second patient;
wherein generating the importance ratio further comprises:
responsive to receipt of the subsequent batch of patient data:
(a) updating the cumulative objective function value for the Input Set based on aggregating the update first and the update second patient objective functions, the first and the second patient objective functions and the one or more per-patient objective functions for first and the second patient previously calculated; and
(b) generating an updated importance ratio weight based on the updated cumulative objective function value, the updated importance ratio representing the Input Set's accuracy with respect to the subsequent batch of patient data, the received batch of patient data and the preceding batches of data;
wherein identifying a most accurate set of estimated input parameters comprises:
determining whether the Input Set is an optimal set of estimated input parameters by comparing the Input Set's updated importance ratio weight with respective current importance ratio weights of other sets of estimated input parameters; and
wherein sending the update set of estimated input parameters comprises:
sending the Input Set to a remote dosing system upon determining the Input Set is the optimal set of estimated input parameters.

22. A computer program product comprising a non-transitory computer-readable medium having a computer-readable program code embodied therein to be executed by one or more processors, the program code including instructions for:
identifying a plurality of sets of estimated input parameters for a pharmacology model;
receiving a first batch of patient data;
generating one or more patient objective function values, each respective patient objective function value representing a confidence measure that a corresponding set of estimated input parameters fed into the pharmacology model returns model output comprising one or more values in the received patient data;
generating an importance ratio for each set of estimated input parameters, each respective importance ratio representing a corresponding set's performance and accuracy with respect to the first batch of data;
identifying a most accurate set of estimated input parameters based on the generated pharmacology model output;
sending an update set of estimated input parameters to a remote dosing model system, the update set of estimated input parameters comprising the identified set of estimated input parameters
receiving, at the remote dosing system, a selection of a type of patient population data and an identification of a particular medication;
executing the one or more prediction dosing models according to the update set of estimated input parameters, the selected type of patient population data and the identified particular medication;
generating, at the remote dosing system, a prediction of at least one behavior of the particular medication at an amount of medication dosage in the selected type of patient population; and
rendering, via a visualization dashboard of the remote dosing system, a graphical visualization of accuracy of the one or more executed prediction dosing models;
wherein generating one or more patient object function values comprises:
calculating a per-patient objective function for each patient identified in a received batch of patient data, the received batch of patient includes observed data of at least a first patient and a second patient, wherein calculating the per-patient objective function comprises:
(i) calculating a first patient objective function value for the first patient representing a first confidence measure of whether a set of estimated input parameters (“Input Set”) fed into a pharmacology model results in returned first model output comprising one or more values of the observed data of the first patient; and
(ii) calculating a second patient objective function value for the second patient representing a second confidence measure of whether the same Input Set fed into the pharmacology model results in returned second model output comprising one or more values of the observed data of the second patient;
wherein generating the importance ratio comprises:
responsive to receipt of the batch of patient data:
(a) calculating a cumulative objective function value for the Input Set based on aggregating the first and the second patient objective functions and any one or more per-patient objective functions for first and the second patient previously calculated due to receipt of one or more preceding batches of data that included earlier observed data of the first and the second patient; and
(b) generating an importance ratio weight based on the cumulative objective function value, the importance ratio weight representing the Input Set's accuracy with respect to the received batch of patient data and the preceding batches of data;
receiving a subsequent batch of patient data that includes subsequent observed data of the first patient and the second patient;
wherein generating one or more patient object function values further comprises:
calculating an update first patient objective function value for the first patient representing an update first confidence measure of whether the Input Set fed into the pharmacology model results in returned first model output comprising one or more values of the subsequent observed data of the first patient;
calculating an update second patient objective function value for the second patient representing an update second confidence measure of whether the same Input Set fed into the pharmacology model results in returned second model output comprising one or more values of the subsequent observed data of the second patient;
wherein generating the importance ratio further comprises:
responsive to receipt of the subsequent batch of patient data:
(a) updating the cumulative objective function value for the Input Set based on aggregating the update first and the update second patient objective functions, the first and the second patient objective functions and the one or more per-patient objective functions for first and the second patient previously calculated; and
(b) generating an updated importance ratio weight based on the updated cumulative objective function value, the updated importance ratio representing the Input Set's accuracy with respect to the subsequent batch of patient data, the received batch of patient data and the preceding batches of data;
wherein identifying a most accurate set of estimated input parameters comprises:
determining whether the Input Set is an optimal set of estimated input parameters by comparing the Input Set's updated importance ratio weight with respective current importance ratio weights of other sets of estimated input parameters; and
wherein sending the update set of estimated input parameters comprises:
sending the Input Set to a remote dosing system upon determining the Input Set is the optimal set of estimated input parameters.

23. A system comprising one or more processors, and a non-transitory computer-readable medium including one or more sequences of instructions that, when executed by the one or more processors, cause the system to perform operations comprising:
identifying a plurality of sets of estimated input parameters for a pharmacology model;
receiving a first batch of patient data;
generating one or more patient objective function values, each respective patient objective function value representing a confidence measure that a corresponding set of estimated input parameters fed into the pharmacology model returns model output comprising one or more values in the received patient data;
generating an importance ratio for each set of estimated input parameters, each respective importance ratio representing a corresponding set's performance and accuracy with respect to the first batch of data;
identifying a most accurate set of estimated input parameters based on the generated pharmacology model output;
sending an update set of estimated input parameters to a remote dosing model system, the update set of estimated input parameters comprising the identified set of estimated input parameters
receiving, at the remote dosing system, a selection of a type of patient population data and an identification of a particular medication;
executing the one or more prediction dosing models according to the update set of estimated input parameters, the selected type of patient population data and the identified particular medication;
generating, at the remote dosing system, a prediction of at least one behavior of the particular medication at an amount of medication dosage in the selected type of patient population; and
rendering, via a visualization dashboard of the remote dosing system, a graphical visualization of accuracy of the one or more executed prediction dosing models;
wherein generating one or more patient object function values comprises:
calculating a per-patient objective function for each patient identified in a received batch of patient data, the received batch of patient includes observed data of at least a first patient and a second patient, wherein calculating the per-patient objective function comprises:
(i) calculating a first patient objective function value for the first patient representing a first confidence measure of whether a set of estimated input parameters (“Input Set”) fed into a pharmacology model results in returned first model output comprising one or more values of the observed data of the first patient; and
(ii) calculating a second patient objective function value for the second patient representing a second confidence measure of whether the same Input Set fed into the pharmacology model results in returned second model output comprising one or more values of the observed data of the second patient;
wherein generating the importance ratio comprises:
responsive to receipt of the batch of patient data:
(a) calculating a cumulative objective function value for the Input Set based on aggregating the first and the second patient objective functions and any one or more per-patient objective functions for first and the second patient previously calculated due to receipt of one or more preceding batches of data that included earlier observed data of the first and the second patient; and
(b) generating an importance ratio weight based on the cumulative objective function value, the importance ratio weight representing the Input Set's accuracy with respect to the received batch of patient data and the preceding batches of data;
receiving a subsequent batch of patient data that includes subsequent observed data of the first patient and the second patient;
wherein generating one or more patient object function values further comprises:
calculating an update first patient objective function value for the first patient representing an update first confidence measure of whether the Input Set fed into the pharmacology model results in returned first model output comprising one or more values of the subsequent observed data of the first patient;
calculating an update second patient objective function value for the second patient representing an update second confidence measure of whether the same Input Set fed into the pharmacology model results in returned second model output comprising one or more values of the subsequent observed data of the second patient;
wherein generating the importance ratio further comprises:
responsive to receipt of the subsequent batch of patient data:
(a) updating the cumulative objective function value for the Input Set based on aggregating the update first and the update second patient objective functions, the first and the second patient objective functions and the one or more per-patient objective functions for first and the second patient previously calculated; and
(b) generating an updated importance ratio weight based on the updated cumulative objective function value, the updated importance ratio representing the Input Set's accuracy with respect to the subsequent batch of patient data, the received batch of patient data and the preceding batches of data;
wherein identifying a most accurate set of estimated input parameters comprises:
determining whether the Input Set is an optimal set of estimated input parameters by comparing the Input Set's updated importance ratio weight with respective current importance ratio weights of other sets of estimated input parameters; and
wherein sending the update set of estimated input parameters comprises:
sending the Input Set to a remote dosing system upon determining the Input Set is the optimal set of estimated input parameters.
